(19) United States
(12) Patent Application Publication
(10) Pub. No.: US 2023/0273202 A1
(43) Pub. Date: Aug. 31, 2023

(54) A METHOD FOR DETERMINING KINETIC PARAMETERS OF A REACTION

(71) Applicant: Creoptix AG, Wädenswil (CH)

(72) Inventor: Kaspar COTTIER, Wädenswil (CH)

(21) Appl. No.: 18/008,155

(22) PCT Filed: Apr. 19, 2021
(86) PCT No.: PCT/IB2021/053195

§ 371 Date: Dec. 2, 2022

(30) Foreign Application Priority Data
Jun. 26, 2020 (CH) 00785/20

(51) Int. Cl. G01N 33/557

(52) U.S. Cl. CPC G01N 33/557

(57) ABSTRACT

According to the present invention there is provided a method for determining the kinetic parameters of a reaction between an analyte and ligands which are attached to a test surface of a flow cell, the method comprising the steps of, (a) flowing a first volume of sample fluid (V1), which contains said analyte, over the test surface, between a first point in time (t₁) to a second point in time (t₂); (b) flowing a first volume of buffer fluid (Vb1), which is without analyte, over the test surface, between a third point in time (t₃) to a fourth point in time (t₄); (c) flowing at least a second volume of sample fluid (V2), which contains said analyte, over the test surface, between a fifth point in time (t₅) to a sixth point in time (t₆); (d) flowing at least a second volume of buffer fluid (Vb2), which is without analyte, over the test surface, between a seventh point in time (t₇) to a eight point in time (t₈); (e) using a sensor to measure the binding of analyte to ligands on the test surface to obtain a binding curve; (f) using the only the parts of the binding curve which are in predefined interval time periods, without using the other parts of the binding curve, to determine the kinetic parameters.

### TECHNICAL DOMAIN

The present invention concerns a method of determining the kinetic parameters of a reaction between an analyte and ligands which are attached to a test surface of a flow cell; and in particular to a method which involves the using substantially less of the parts of the binding curve which represent association of the analyte to the ligands, and rather using mainly the parts of a binding curve which represent dissociation of the analyte from the ligands, in order to determine the kinetic parameters.

### BACKGROUND TO THE INVENTION

In existing methods for determining the kinetic parameters of a reaction between an analyte and ligands which are attached to a test surface of a flow cell, volumes of sample fluids containing said analyte are consecutively flowed over the test surface. As the volumes of sample fluids containing said analyte are consecutively flowed over the test surface, a sensor is used to measure the amount of analyte which is bound to the ligands on the test surface; a curve, representing the amount of analyte which is bound to the ligands on the test surface, known as a binding curve, is output by the sensor. The binding curve will contain association phases and disassociation phases. An association phase occurs when a volume of sample fluid is passing over the test surface and analyte in the sample fluid is binding to the ligands; the binding curve will show an increase in the amount of analyte which is bound to the ligands on the test surface during the association phase. A dissociation phase occurs after a volume of sample fluid is passing over the test surface, and before the next volume of sample fluid is flowed over the test surface; the binding curve will show a decrease in the amount of analyte which is bound to the ligands on the test surface during the dissociation phase and the previously bound analytes become dissociated (or detached) from the ligands.

The entire binding curve (i.e. all of the association phases and disassociation phases) is then used to determine the kinetic parameters of the reaction between the analyte and ligands on the test surface; typically this is done by identifying a predefined model, which has known kinetic parameters, which best fits to the binding curve; the kinetic parameters of the model which best fits to the binding curve are then considered to be the kinetic parameters of the reaction.

However, the problem with using the entire binding curve to determine the kinetic parameters of the reaction between the analyte and ligands on the test surface, is that it may lead to inaccurate determination of the kinetic parameters of the reaction: existing sensors which are used to measure the amount of analyte which is bound to the ligands on the test surface and output a binding curve, are sensitive to changes in refractive index of the volumes of sample fluid; when a volume of sample fluid containing analyte is flowed over the test surface, changes in the refractive index of the volume of sample fluid can introduce artefacts in the binding curve; these artefacts are present in the association phases of the binding curve; because the entire binding curve (i.e. all of the association phases and disassociation phases) is then used to determine the kinetic parameters of the reaction between the analyte and ligands on the test surface, these artefacts in turn leads to inaccurate determination of the kinetic parameters of the reaction.

It is an aim of the present invention to obviate, or mitigate, at least some of the disadvantages associated with the existing methods in the field. In particular it is an aim of the present invention to provide an improve method for determining the kinetic parameters of a reaction, which is less vulnerable to artefacts in the binding curve created by refractive index change which occur when a volume of sample fluid is flowed over the test surface of a flow cell.

### SUMMARY OF THE INVENTION

According to the present invention, this aim is achieved by a method having, at least, the steps recited in claim 1. The dependent claims recite favourable, optional, steps which can be performed in various embodiments of the invention.

Advantageously, in the method of the present invention mainly the dissociation phase of the binding curve (i.e. the part of the binding curve corresponding to the period after a volume of sample fluid has been flowed over the test surface, an before the next volume of sample fluid is flowed over the test surface), and not the association phase of binding curve (i.e. the part of the binding cure corresponding to when a volume of sample fluid is flowing over the test surface and analyte in the sample fluid is binding to the ligands), is used to determine kinetic parameters of the reaction.

In particular in a first embodiment only the dissociation phases of the binding curve and no association phase of binding curve are used to determine the kinetic parameters; and in a second embodiment the dissociation phases of the binding curve and only a very small part of the association phases of binding curve are used to determine the kinetic parameters. In the second embodiment the small part of the association phases of the binding curve can be so small that artefacts in those parts of the binding curve have a negligible effect on the determination of the kinetic parameters. In either embodiment the amount of the association phases of the binding curve which is used to determine the kinetic parameters is reduced compared to the prior art.

Accordingly, in the present invention, artefacts in the binding curve created by refractive index changes which occur when a volume of sample fluid is flowed over the test surface of a flow cell, will have less of an effect on the determination of the kinetic parameters.

### BRIEF DESCRIPTION OF THE DRAWINGS

Exemplary embodiments of the invention are disclosed in the description and illustrated by the drawings in which:

FIG. 1 is a complete binding curve which comprises six association phases and six dissociation phases, which resulted from having flowed, consecutively, respective six volumes of sample fluid containing analyte, over the test surface of the flow cell;

FIG. 2 shows the parts of the binding curve of FIG. 1 which are in a plurality of predefined interval time periods;

FIG. 3 shows a normalized concentration curve which is used to determine said plurality of predefined interval time periods.

### DETAILED DESCRIPTION OF EMBODIMENTS OF THE PRESENT INVENTION

According to the present invention there is provided a method for determining the kinetic parameters of a reaction between an analyte and ligands which are attached to a test surface of a flow cell. The method comprises the steps of:

(a) Flowing a first volume of sample fluid (V1), which contains said analyte, over the test surface, between a first point in time (t₁) to a second point in time (t₂).

(b) flowing a first volume of buffer fluid (Vb1) (which is without analyte), over the test surface, between a third point in time (t₃) to a fourth point in time (t₄) (the third point in time (t₃) may be equal to the second point in time (t₂)); most preferably dissociation of at least some analytes from the first volume of sample fluid (V1) which were bound to the ligands on the test surface occurs between the third point in time (t₃) to the fourth point in time (t₄). In one embodiment the buffer fluid is configured to promote the dissociation of bound analyte from the ligands.

(c) Flowing at least a second volume of sample fluid (V2), which contains said analyte, over the test surface, between a fifth point in time (t₅) to a sixth point in time (t₆) (the fifth point in time (t₅) may be equal to the fourth point in time (t₄));

(d) flowing a second volume of buffer fluid (Vb2) (which is without analyte), over the test surface, between a seventh point in time (t₇) to a eight point in time (t₈) (the seventh point in time (t₇) may be equal to the sixth point in time (t₆)); most preferably dissociation of at least some analytes from the second volume of sample fluid (V2), which were bound to the ligands on the test surface, occurs between the seventh point in time (t₇) and eight point in time (t₈). In one embodiment the buffer fluid is configured to promote the dissociation of bound analyte from the ligands.

(e) Using a sensor to measure the binding of analyte to ligands on the test surface to obtain a binding curve.

(f) Using only the parts of the binding curve which are in predefined interval time periods, without using the other parts of the binding curve, to determine the kinetic parameters.

In order to determine the kinetic parameters only the parts of the binding curve which are predefined interval time periods, the following steps are carried out: establishing a normalized concentration curve (c(t)) which describes the concentration of the analyte at the test surface over time; estimating values for the kinetic parameters Rmax, kₐ, k_{d}, wherein Rmax is a predefined theoretical maximum binding curve value corresponding to a saturation of the ligands, such as for instance when all binding sites of the ligands are occupied by analyte bound to the ligands, kₐ is the association rate constant and k_{d} is the dissociation rate constant; using said normalized concentration curve (c(t)), and said estimated values for the kinetic parameters Rmax, kₐ, k_{d}, to solve the differential reaction equation:

$$\frac{d R}{d t} = {k}_{a} c \left(t\right) \left({R}_{m a x} - R \left(t\right)\right) - {k}_{d} R \left(t\right)$$

in order to obtain a simulated binding curve sbc = R(t).

Once the differential reaction equation has been obtained, then extracting the parts of the simulated binding curve which are in said predefined interval time periods (tjΔ) to provide respective partial simulated binding curves (sbcj).

A partial chi square (χ²) of the simulated binding curve is then established according to the following equation:

$${\text{χ}}^{2} = {\sum }_{j} {\sum }_{i} \frac{{\left({\text{sbcj}}_{i} {\text{-dmbj}}_{i}\right)}^{2}}{{N}_{d m b j}}$$

wherein dmbj is the part of the binding curve which is in the j-th predefined interval time period (tjΔ), and wherein sbcj is the j-th partial simulated binding curve and N_{dmbj} is the number of data points in the part of the binding curve which is in the j-th predefined interval time period (tjΔ).

After the partial chi square (χ²) of the simulated binding curve has been determined then, minimizing said determined partial chi square (χ²), wherein the values of said kinetic parameters Rmax, kₐ, k_{d} which minimize said determined partial chi square (χ²) define the kinetic parameters of said reaction between the analyte and ligands which are attached to a test surface of the flow cell.

In the preferred embodiment the step of estimating values for the kinetic parameters Rmax, kₐ, k_{d}, comprises using a Levenberg-Marquard algorithm to find the kinetic parameters which minimize the partial chi square (χ²).

In a further preferred embodiment the step of estimating values for the kinetic parameters Rmax, kₐ, k_{d}, comprises using Estimators to find the kinetic parameters which minimize the partial chi square (χ²).

In the preferred embodiment the method comprises the steps of using a sensor to measure the binding of analyte to ligands on the test surface, continuously from the first point in time (t₁) to the ninth point in time (t₉) and then extracting the parts of the binding curve which are in said predefined interval time periods; and using only said extracted parts of the binding curve to determine the kinetic parameters.

In another embodiment the method comprises the steps of using a sensor to measure the binding of analyte to ligands on the test surface, continuously between from the first point in time (t₁) to the ninth point in time (t₉); zeroing the parts of the binding curve which are outside of said predefined interval time periods; and then using only said parts of the binding curve which are not zeroed to determine the kinetic parameters.

### Exemplary Embodiment 1

In the first embodiment the predefined interval time periods are a first and second interval time periods (t1Δ, t2Δ); wherein the first interval time period (t1Δ) occurs between the second point in time (t₂) and the fifth point in time (t₅); and wherein the second interval time period (t2Δ) occurs between the sixth point in time (t₆) and a ninth point in time (t₉) wherein the ninth point in time (t₉) occurs sometime after the eight point in time (t₈) (or, in another embodiment, the ninth point in time (t₉) is equal to the eight point in time (t₈)). Advantageously, in this embodiment only the parts of the binding curve which correspond to the dissociation phase are used to determine the kinetic parameters.

In one embodiment the first interval time period (t1Δ) is a portion of the duration of time between the second point in time (t₂) and the fifth point in time (t₅); and the second interval time period (t2Δ) is a portion of the duration of time between the sixth point in time (t₆) and a ninth point in time (t₉) wherein the ninth point in time (t₉) occurs sometime after the eight point in time (t₈) (or, in another embodiment, the ninth point in time (t₉) is equal to the eight point in time (t₈)). For example the first interval time period (t1Δ) may be between a predefined time period after the second point in time (t₂), until the fifth point in time (t₅), or until a predefined time period before the fifth point in time (t₅); and the second interval time period (t2Δ) may be between a predefined time period after the sixth point in time (t₆) until the eight point in time (t₈), or until a predefined time period before the eight point in time (t₈). In another embodiment the first interval time period (t1Δ) is defined by the whole time interval between the second point in time (t₂) and the fifth point in time (t₅); and the second interval time period (t2Δ) is defined by the whole time interval between the sixth point in time (t₆) and a ninth point in time (t₉) wherein the ninth point in time (t₉) occurs sometime after the eight point in time (t₈) (or, in another embodiment, the ninth point in time (t₉) is equal to the eight point in time (t₈)).

This first embodiment comprises the steps of:

(a) Flowing a first volume of sample fluid (V1), which contains said analyte, over the test surface, between a first point in time (t₁) to a second point in time (t₂).

(b) flowing a first volume of buffer fluid (Vb1) (which is without analyte), over the test surface, between a third point in time (t₃) to a fourth point in time (t₄) (the third point in time (t₃) may be equal to the second point in time (t₂)); most preferably dissociation of at least some analytes from the first volume of sample fluid (V1) which were bound to the ligands on the test surface occurs between the third point in time (t₃) to the fourth point in time (t₄). In one embodiment the buffer fluid is configured to promote the dissociation of bound analyte from the ligands.

(c) Flowing at least a second volume of sample fluid (V2), which contains said analyte, over the test surface, between a fifth point in time (t₅) to a sixth point in time (t₆) (the fifth point in time (t₅) may be equal to the fourth point in time (t₄));

(d) flowing a second volume of buffer fluid (Vb2) (which is without analyte), over the test surface, between a seventh point in time (t₇) to a eight point in time (t₈) (the seventh point in time (t₇) may be equal to the sixth point in time (t₆)); most preferably dissociation of at least some analytes from the second volume of sample fluid (V2), which were bound to the ligands on the test surface, occurs between the seventh point in time (t₇) and eight point in time (t₈). In one embodiment the buffer fluid is configured to promote the dissociation of bound analyte from the ligands.

(e) Using a sensor to measure the binding of analyte to ligands on the test surface, at least during a first and a second interval time period (t1Δ, t2Δ), wherein the first interval time period (t1Δ) occurs between the second point in time (t₂) and fifth point in time (t₅), and the second interval time period (t2Δ) occurs between the sixth point in time (t₆) and a ninth point in time (t₉) wherein the ninth point in time (t₉) occurs sometime after the eight point in time (t₈) (or, in another embodiment, the ninth point in time (t₉) is equal to the eight point in time (t₈)) to obtain a binding curve. In one embodiment the first interval time period (t1Δ) is a portion of the duration of time between the second point in time (t₂) and the fifth point in time (t₅); and the second interval time period (t2Δ) is a portion of the duration of time between the sixth point in time (t₆) and a ninth point in time (t₉) wherein the ninth point in time (t₉) occurs sometime after the eight point in time (t₈) (or, in another embodiment, the ninth point in time (t₉) is equal to the eight point in time (t₈)). For example the first interval time period (t1Δ) may be between a predefined time period after the second point in time (t₂), until the fifth point in time (t₅), or until a predefined time period before the fifth point in time (t₅); and the second interval time period (t2Δ) may be between a predefined time period after the sixth point in time (t₆) until the eight point in time (t₈), or until a predefined time period before the eight point in time (t₈). In another embodiment the first interval time period (t1Δ) is defined by the whole time interval between the second point in time (t₂) and the fifth point in time (t₅); and the second interval time period (t2Δ) is defined by the whole time interval between the sixth point in time (t₆) and a ninth point in time (t₉) wherein the ninth point in time (t₉) occurs sometime after the eight point in time (t₈) (or, in another embodiment, the ninth point in time (t₉) is equal to the eight point in time (t₈)).

(f) Using the part of the binding curve which is in said first and second interval time periods (t1Δ, t2Δ), without using the parts of the binding curve which are between a first point in time (t₁) and second point in time (t₂ₐ) and between the fifth point in time (t₅) to the sixth point in time (t₆), to determine the kinetic parameters.

In this first embodiment substantially none of the binding curve which are between a first point in time (t₁) and second point in time (t₂) and between the fifth point in time (t₅) and sixth point in time (t₆) are used to determine the kinetic parameters. Accordingly, artefacts in the binding curve created by refractive index changes which occur when a volume of sample fluid is flowed over the test surface of a flow cell, will have less of an effect on the determination of the kinetic parameters.

In one embodiment the method comprises the steps of using a sensor to measure the binding of analyte to ligands on the test surface, continuously from the first point in time (t₁) to the ninth point in time (t₉) and then extracting the parts of the binding curve which are in said first and second interval time periods (t1Δ, t2Δ); and using only said extracted parts of the binding curve to determine the kinetic parameters.

In another embodiment the method comprises the steps of using a sensor to measure the binding of analyte to ligands on the test surface, continuously between from the first point in time (t₁) to the ninth point in time (t₉); zeroing the parts of the binding curve which are outside of said first and second interval time periods (t1Δ, t2Δ); and then using only said parts of the binding curve which are not zeroed to determine the kinetic parameters.

The step of using the part of the binding curve which is in said first and second interval time periods (t1Δ, t2Δ), without using the parts of the binding curve which are between a first point in time (t₁) and second point in time (t₂) and between the fifth point in time (t₅) and sixth point in time (t₆), to determine the kinetic parameters, can be carried out using any known techniques for determining kinetic parameters using a binding curve; those very same techniques can be used in the present invention to determine kinetic parameters, the difference being the techniques are applied to the parts of the binding curve in said first and second interval time periods (t1Δ, t2Δ), and not to the parts of the binding curve which are between a first point in time (t₁) and second point in time (t₂) and between the fifth point in time (t₅) and sixth point in time (t₆).

In the most favourable embodiment the step of using the parts of the binding curve which is in said first and second interval time periods (t1Δ, t2Δ), without using the parts of the binding curve which are between a first point in time (t₁) and second point in time (t₂) and between the fifth point in time (t₅) and sixth point in time (t₆), to determine the kinetic parameters, preferably comprises the steps of: establishing a normalized concentration curve (c(t)) which describes the concentration of the analyte at the test surface over time (preferably a concentration curve is first established then it is normalized to provide said normalized concentration curve (c(t))); estimating values for the kinetic parameters Rmax, kₐ, k_{d}, wherein Rmax is a predefined theoretical maximum binding curve value corresponding to a saturation of the ligands corresponding to a saturation of the ligands, such as for instance when all binding sites of the ligands are occupied by analyte bound to the ligands, kₐ is the association rate constant and k_{d} is the dissociation rate constant; using said normalized concentration curve (c(t)), and said estimated values for the kinetic parameters Rmax, kₐ, k_{d}, to solve the differential reaction equation:

$$\frac{d R}{d t} = {k}_{a} c \left(t\right) \left({R}_{m a x} - R \left(t\right)\right) - {k}_{d} R \left(t\right)$$

in order to obtain a simulated binding curve sbc = R(t).

Once the differential reaction equation has been obtained, then extracting the part of the simulated binding curve which is in said first interval time period (t1Δ) to provide a first partial simulated binding curve (sbc1); and extracting the part of the simulated binding curve which is in said second interval time period (t2Δ) to provide a second partial simulated binding curve (sbc2); and determining a partial chi square (χ²) of the simulated binding curve according to the following equation:

$${\text{χ}}^{2} = \sum \frac{{\left(\text{sbc1} {}_{i} - {\text{dmb1}}_{i}\right)}^{2}}{{N}_{d m b 1}} + \sum \frac{{\left({\text{sbc2}}_{i} - {\text{dmb2}}_{i}\right)}^{2}}{{N}_{d m b 2}}$$

wherein dmb1 is the part of the binding curve which is in said first interval time period (t1Δ) and dmb2 is the part of the binding curve which is in said second interval time period (t1Δ), and wherein sbc1 is the first partial simulated binding curve and sbc2 is the second partial simulated binding curve, and wherein N_{dmb1} is the number of data points of the binding curve which is in said first interval time period (t1Δ) and N_{dmb2} is the number of data points of the binding curve which is in said second interval time period (t2Δ).

After the partial chi square (χ²) of the simulated binding curve has been determined then, minimizing said determined partial chi square (χ²), wherein the values of said kinetic parameters Rmax, kₐ, k_{d} which minimize said determined partial chi square (χ²) define the kinetic parameters of said reaction between the analyte and ligands which are attached to a test surface of the flow cell.

In the preferred embodiment the step of estimating values for the kinetic parameters Rmax, kₐ, k_{d}, comprises using a Levenberg-Marquard algorithm to find the kinetic parameters which minimize the partial chi square (χ²).

In a further preferred embodiment the step of estimating values for the kinetic parameters Rmax, kₐ, k_{d}, comprises using Estimators to find the kinetic parameters which minimize the partial chi square (χ²).

### Exemplary Embodiment 2

In the first embodiment described above, the predefined interval time periods comprise first and second interval time periods (t1Δ, t2Δ); wherein the first interval time period (t1Δ) occurs between the second point in time (t₂) and fifth point in time (t₅); and wherein the second interval time period (t2Δ) occurs between the sixth point in time (t₆) and a ninth point in time (t₉) wherein the ninth point in time (t₉) occurs sometime after the eight point in time (t₈) (or, in another embodiment, the ninth point in time (t₉) is equal to the eight point in time (t₈)). In this second embodiment, however, the predefined interval time periods are determined from a concentration curve (or from a normalized concentration curve). The second embodiment comprises the steps of:

(a) Flowing a first volume of sample fluid (V1), which contains said analyte, over the test surface, between a first point in time (t₁) to a second point in time (t₂).

(b) flowing a first volume of buffer fluid (Vb1) (which is without analyte), over the test surface, between a third point in time (t₃) to a fourth point in time (t₄) (the third point in time (t₃) may be equal to the second point in time (t₂)); most preferably dissociation of at least some analytes from the first volume of sample fluid (V1) which were bound to the ligands on the test surface occurs between the third point in time (t₃) to the fourth point in time (t₄). In one embodiment the buffer fluid is configured to promote the dissociation of bound analyte from the ligands.

(c) Flowing at least a second volume of sample fluid (V2), which contains said analyte, over the test surface, between a fifth point in time (t₅) to a sixth point in time (t₆) (the fifth point in time (t₅) may be equal to the fourth point in time (t₄));

(d) flowing a second volume of buffer fluid (Vb2) (which is without analyte), over the test surface, between a seventh point in time (t₇) to a eight point in time (t₈) (the seventh point in time (t₇) may be equal to the sixth point in time (t₆)); most preferably dissociation of at least some analytes from the second volume of sample fluid (V2), which were bound to the ligands on the test surface, occurs between the seventh point in time (t₇) and eight point in time (t₈). In one embodiment the buffer fluid is configured to promote the dissociation of bound analyte from the ligands.

(e) Using a sensor to measure the binding of analyte to ligands on the test surface, to obtain a binding curve.

(f) Using the parts of the binding curve which is in a predefined first interval time periods (t1Δ) and second interval time period (t2Δ), to determine the kinetic parameters.

In this second embodiment substantially less of the parts of the binding curve which are between a first point in time (t₁) and second point in time (t₂) and between the fifth point in time (t₅) and sixth point in time (t₆) are used to determine the kinetic parameters. Accordingly, artefacts in the binding curve created by refractive index changes which occur when a volume of sample fluid is flowed over the test surface of a flow cell, will have less of an effect on the determination of the kinetic parameters.

In one embodiment the method comprises the steps of using a sensor to measure the binding of analyte to ligands on the test surface, continuously from the first point in time (t₁) to the ninth point in time (t₉) and then extracting the parts of the binding curve which are in said first and second interval time periods (t1Δ, t2Δ); and using only said extracted parts of the binding curve to determine the kinetic parameters.

In another embodiment the method comprises the steps of using a sensor to measure the binding of analyte to ligands on the test surface, continuously between from the first point in time (t₁) to the ninth point in time (t₉); zeroing the parts of the binding curve which are outside of said first and second interval time periods (t1Δ, t2Δ); and then using only said parts of the binding curve which are in said first and second interval time periods (t1Δ, t2Δ) (i.e. the parts of the binding curve which are not zeroed) to determine the kinetic parameters.

The second embodiment further comprises a calibration step which may be carried out before steps (a)-(f), or, may be carried out after steps (a)-(e) (before step (f) is carried out), to determine the first and second interval time periods (t1Δ, t2Δ).

In one embodiment the calibration step comprises, establishing a concentration curve which describes the concentration of the analyte at the test surface over time, and then normalizing that concentration curve to provide a normalized concentration curve (c(t)); and then using that normalized concentration curve (c(t)) to determine the first and second interval time periods (t1Δ, t2Δ). In another embodiment the calibration step comprises, establishing a concentration curve which describes the concentration of the analyte at the test surface over time; then using that concentration curve to determine the first and second interval time periods (t1Δ, t2Δ). Also it should be noted that the same steps are carried out to determine the first and second interval time periods (t1Δ, t2Δ) from the normalized concentration curve (c(t)) as are carried out to determine the first and second interval time periods (t1Δ, t2Δ) from the concentration curve (c(t)). In both variations the normalized concentration curve (c(t)) is used to determine the kinetic parameters.

In the present exemplary second embodiment it will be described establishing a concentration curve which describes the concentration of the analyte at the test surface over time, and then normalizing that concentration curve to provide a normalized concentration curve (c(t)); and then using that normalized concentration curve (c(t)) to determine the first and second interval time periods (t1Δ, t2Δ):

In the present exemplary second embodiment in which first and second volumes of sample fluid (V1,V2) are flowed over the test surface of the flow cell in order to establish a binding curve, a first volume of refractive index standard fluid (V1′), which contains a known concentration of a reference molecule, is flowed over the test surface between a first reference point in time (t′₁) to a second reference point in time (t′₂).

Importantly the rate at which the first volume of refractive index standard fluid (V1′) is flowed over the test surface (i.e. the flow rate) is equal to the rate at which the first volume of sample fluid (V1) is flowed over the test surface; also the duration of time between the a first reference point in time (t′₁) to a second reference point in time (t′₂) is equal to the duration of time between the first point in time (t₁) to a second point in time (t₂).

After the first volume of refractive index standard fluid (V1′), which contains a known concentration of a reference molecule, has been flowed over the test surface, a first volume of buffer fluid (Vb1′) (which is without reference molecules) is flowed over the test surface, between a third reference point in time (t′₃) to a fourth reference point in time (t′₄) (the third reference point in time (t′₃) may be equal to the second reference point in time (t′₂)).

A second volume of refractive index standard fluid (V2′), which contains a known concentration of a reference molecule, is flowed then over the test surface between a fifth reference point in time (t′₅) to a sixth reference point in time (t′₆).

Importantly the rate at which the second volume of refractive index standard fluid (V2′) is flowed over the test surface (i.e. the flow rate) is equal to the rate at which the second volume of sample fluid (V2) is flowed over the test surface; also the duration of time between the fifth reference point in time (t′₅) to a sixth reference point in time (t′₆) is equal to the duration of time between the fifth point in time (t₅) to a sixth point in time (t₆); also the ratio of the concentration of the second volume of refractive index standard fluid (V2′) to the concentration of the first volume of refractive index standard fluid (V1′), is equal to the ratio of the concentration of the second volume of sample fluid (V2) to first volume of sample fluid (V1); also the duration of time between the second reference point in time (t′₂) and the fifth reference point in time (t′₅) is equal to the duration of time between the second point in time (t₂) and the fifth reference point in time (t′₅).

After the second volume of refractive index standard fluid (V2′), which contains a known concentration of a reference molecule, has been flowed over the test surface flowing, a second volume of buffer fluid (Vb2′) (which is without reference molecules) is flowed over the test surface, between a seventh reference point in time (t₇) to a eight reference point in time (t₈) (the seventh point in time (t₇) may be equal to the sixth reference point in time (t′₆)).

The concentration of the reference molecule at the test surface is measured continuously over the period from the first reference point in time (t′₁) to a ninth reference point in time (t′₉) wherein the ninth reference point in time (t′₉) occurs sometime after the eight reference point in time (t′₈) (or, in another embodiment, the ninth reference point in time (t′₉) is equal to the eight reference point in time (t′₈)), so as to obtain a concentration curve (cmc).

The concentration curve (cmc) is then normalized by dividing the concentration curve (cmc) by the maximum value of the concentration curve (max(cmc)), and multiplying the result by a known maximum concentration (cₘₐₓ) of analyte in the in the first and second volumes of sample fluid (V1,V2)), as illustrated in the following equation:

$$\text{c} \left(\text{t}\right) = \left(\left(\text{cmc}\right) / \text{max} \left(\text{cmc}\right)\right) * {\text{c}}_{max}$$

to obtain a normalized concentration curve (c(t)).

In this example the concentration (c(V1)) of analyte in the first volume of sample fluid (V1) is equal to the concentration (c(V2)) of analyte in the second volume of sample fluid (V2) (i.e. both first and second volumes of sample fluid (V1,V2) have the same concentration of analyte); if however one of the volumes of sample fluid (V1,V2) would contain a higher concentration of analyte than the other, then the higher concentration value would define cₘₐₓ (i.e. (cₘₐₓ = max (c(V1), c(V2)). It should be understood that the concentration of analyte in a volume of sample fluid can be determined using any suitable technique which is well known in the art.

In order to determine the first and second interval time periods (t1Δ, t2Δ) using the normalized concentration curve (c(t)), a threshold concentration is selected. Most preferably the threshold concentration is determined as a predefined percentage of the maximum value of the normalized concentration curve (c(t)); for example if the maximum value of the normalized concentration curve (c(t)) curve is ‘200’, then the threshold concentration may be selected to be 5% of the maximum value, which in this example would be 5%*200 = 10; so the threshold concentration in this example would be ‘10’; in another example the threshold concentration may be 2% of the maximum value, which in this example would be 2%*200 = 4, so the threshold concentration in this example would be ‘4’.

The earliest time interval on the normalized concentration curve (c(t)), from the time when the normalized concentration curve (c(t)) drops below the threshold concentration, until the time when the normalized concentration curve (c(t)) is equal to the threshold concentration, defines the first interval time period (t1Δ). The next time interval on the normalized concentration curve (c(t)) (which occurs sometime after the first interval time period (t1Δ)), from the time when the normalized concentration curve (c(t)) drops below the threshold concentration, until the ninth reference point in time (t′₉), defines the second interval time period (t2Δ).

It should be understood that in a variation of this embodiment the concentration curve (not normalized) may alternatively be used to determine the first and second interval time periods (t1Δ, t2Δ); again in this variation of the embodiment a threshold concentration is selected. Most preferably the threshold concentration is determined as a predefined percentage of the maximum value of the concentration curve; for example if the maximum value of the concentration curve curve is ‘2000’, then the threshold concentration may be selected to be 5% of the maximum value, which in this example would be 5%*2000 = 100; so the threshold concentration in this example would be ‘100’; in another example the threshold concentration may be 2% of the maximum value, which in this example would be 2%*2000 = 40, so the threshold concentration in this example would be ‘40. The earliest time interval on the concentration curve, from the time when the concentration curve drops below the threshold concentration, until the time when the concentration curve is equal to the threshold concentration, defines the first interval time period (t1Δ). The next time interval on the concentration curve (which occurs sometime after the first interval time period (t1Δ)), from the time when the concentration curve drops below the threshold concentration, until the ninth reference point in time (t′₉), defines the second interval time period (t2Δ).

In the above examples, most preferably the refractive index standard fluid preferably comprises a buffer fluid which contains a known concentration of a reference molecule; and the reference molecule (which is present in a known concentration in the refractive index standard fluid) may comprise, for example, DMSO or Glucose. Most preferably the concentration of a reference molecule in the refractive index standard fluid (and thus the concentration of a reference molecule in the first volume of refractive index standard fluid (V1′), and also the concentration of a reference molecules in the second volume of refractive index standard fluid (V2′)) is preferably 0.1% v/v, or 0.5% v/v, or 1% v/v, so the refractive index of the refractive index standard fluid is different from the refractive index of first and second volumes of buffer fluid (Vb1′,Vb2′) which are without a reference molecules.

The step (f) of using the part of the binding curve which is in said first and second interval time periods (t1Δ, t2Δ), to determine the kinetic parameters, can be carried out using any known techniques for determining kinetic parameters using a binding curve; the very same techniques can be used in the present invention to determine kinetic parameters, the difference being the techniques are applied to the parts of the binding curve in said first and second interval time periods (t1Δ, t2Δ).

In the most favourable embodiment the step of using the parts of the binding curve which is in said first and second interval time periods (t1Δ, t2Δ), to determine the kinetic parameters, preferably comprises the steps of, establishing a normalized concentration curve (c(t)) which describes the concentration of the analyte at the test surface over time (if a normalized concentration curve (c(t)) has not already been established; for example the normalized concentration curve (c(t) established in the calibration step may be used) (using any of steps described in the present application for obtaining a normalized concentration curve (c(t))); estimating values for the kinetic parameters Rmax, kₐ, k_{d}, wherein Rmax is a predefined theoretical maximum binding curve value corresponding to a saturation of the ligands, such as for instance when all binding sites of the ligands are occupied by analyte bound to the ligands, kₐ is the association rate constant and k_{d} is the dissociation rate constant; using said normalized concentration curve (c(t)) determined in said calibration step, and said estimated values for the kinetic parameters Rmax, kₐ, k_{d}, to solve the differential reaction equation:

$$\frac{d R}{d t} = {k}_{a} c \left(t\right) \left({R}_{m a x} - R \left(t\right)\right) - {k}_{d} R \left(t\right)$$

in order to obtain a simulated binding curve sbc = R(t).

Once the differential reaction equation has been obtained, then extracting the part of the simulated binding curve which is in said first interval time period (t1Δ) to provide a first partial simulated binding curve (sbc1); and extracting the part of the simulated binding curve which is in said second interval time period (t2Δ) to provide a second partial simulated binding curve (sbc2); and determining a partial chi square (χ²) of the simulated binding curve according to the following equation:

$${\text{χ}}^{2} = \sum \frac{{\left(\text{sbc1} {}_{i} - {\text{dmb1}}_{i}\right)}^{2}}{{N}_{d m b 1}} + \sum \frac{{\left({\text{sbc2}}_{i} - {\text{dmb2}}_{i}\right)}^{2}}{{N}_{d m b 2}}$$

wherein dmb1 is the part of the binding curve which is in said first interval time period (t1Δ) and dmb2 is the part of the binding curve which is in said second interval time period (t1Δ), and wherein sbc1 is the first partial simulated binding curve and sbc2 is the second partial simulated binding curve, and wherein N_{dmb1} is the number of data points of the binding curve which is in said first interval time period (t1Δ) and N_{dmb2} is the number of data points of the binding curve which is in said second interval time period (t2Δ).

After the partial chi square (χ²) of the simulated binding curve has been determined then, minimizing said determined partial chi square (χ²), wherein the values of said kinetic parameters Rmax, kₐ, k_{d} which minimize said determined partial chi square (χ²) define the kinetic parameters of said reaction between the analyte and ligands which are attached to a test surface of the flow cell.

In the preferred embodiment the step of estimating values for the kinetic parameters Rmax, kₐ, k_{d}, comprises using a Levenberg-Marquard algorithm to find the kinetic parameters which minimize the partial chi square (χ²).

In a further preferred embodiment the step of estimating values for the kinetic parameters Rmax, kₐ, k_{d}, comprises using Estimators to find the kinetic parameters which minimize the partial chi square (χ²).

### Establishing a Normalized Concentration Curve

Each of the embodiments of the present invention involve a step of establishing a concentration curve which describes the concentration of the analyte at the test surface over time. It should be noted that the concentration curve may be established using any suitable known methods in the art.

In the present invention the step of establishing a concentration curve may be done using volumes of refractive index standard fluid, which contains known concentration of a reference molecule, as describe in the second embodiment above.

In another embodiment the concentration curve which describes the concentration of the analyte at the test surface over time, can be established using a mathematical model of the convection and diffusion fluid dynamics transport phenomena.

In yet another embodiment the concentration curve which describes the concentration of the analyte at the test surface over time, can be established using a simplified model not considering transport phenomena: In this embodiment the concentration curve formed by setting the concentration curve to a ‘zero’ value for time periods before the first point in time (t₁), during the first interval time period (t1Δ), and during the second interval time period (t2Δ); the concentration curve set to a constant value of c₁ between the first point in time (t₁) and the second point in time (t₂), wherein c₁ is the concentration of analyte at the test surface between the first point in time (t₁) and the second point in time (t₂) as the first volume of sample fluid (V1) is flowed over the test surface (i.e. c₁ is the concentration of the analyte in the first volume of sample fluid); the concentration curve set to a constant value of c₂ between the fifth point in time (t₅) to a sixth point in time (t₆), wherein c₂ is the average concentration of analyte at the test surface between the fifth point in time (t₅) and the sixth point in time (t₆), as the second volume of sample fluid (V2) is flowed over the test surface (i.e.c₂ is he concentration of the analyte in the first volume of sample fluid

Regardless of what steps are taken to establish the concentration curve, in each embodiment of the present invention, after the concentration curve has been established the concentration curve is then normalized to provide a normalized concentration curve (c(t)); and then that normalized concentration curve (c(t)) is used to determine kinetic parameters in the same manner as described in the embodiment above.

It should be understood that in some embodiments (e.g. the second embodiment) the concentration curve, or the normalized concentration curve, may be further used to determine the predefined interval time periods on the binding curve which are used to determine the kinetic parameters.

### Exemplary Embodiment With Multiple Volumes of Samples Fluids

Importantly, the above examples (in particular the first and second embodiments) describe flowing first and second volumes of sample fluid (V1,V2) containing analyte which, over the test surface of the flow cell. However, it should be understood that any number of volumes of sample fluid can be flowed over the test surface of the flow cell (with respective volumes of buffer fluid flowed over the test surface between volumes of sample fluid); the same principles of the present invention apply when multiple volumes of sample fluid (e.g. more than two) are flowed over the test surface of the flow cell. With respect to the second embodiment, during the calibration step, the number of volumes of refractive index standard fluid which are flowed over the test surface of the flow cell, corresponds to the number of volumes of sample fluid containing analyte which, have been, or are to be, flowed over the test surface of the flow cell.

Accordingly, the method of the present invention may comprise the steps of, consecutively flowing a plurality of flowing a plurality of volumes of sample fluid, each of which contains said analyte, over the test surface, wherein there is an interval time period between each respective volume of sample fluid is flowed over the test surface; and flowing a respective volume of buffer fluid (which is without analyte) over the test surface during each respective interval time period; using a sensor to measure the binding of analyte to ligands on the test surface, to obtain a binding curve; using only parts of the binding curve which are in a plurality of predefined interval time periods, to determine the kinetic parameters.

The plurality of predefined interval time periods may be determined in the manner described in first and second embodiments.

As was the case for the first and second embodiments, the method may comprise steps of, using a sensor to measure the binding of analyte to ligands on the test surface, continuously, between the time when the first volume of sample fluid is flowed over the test surface, until the time when the last volume of buffer fluid is flowed over the test surface; extracting the parts of the binding curve which are in said plurality of predefined interval time periods ; and using only said extracted parts of the binding curve to determine the kinetic parameters. In a further embodiment the method comprises extracting the parts of the binding curve which is in said are in said plurality of predefined interval time periods and the part of the binding curve which is after the last volume of sample fluid has flowed over the test surface until the level of binding drops to a predefined threshold level; and using only said extracted parts of the binding curve to determine the kinetic parameters.

Alternatively, said method may comprise the steps of, using a sensor to measure the binding of analyte to ligands on the test surface, continuously, between the time when the first volume of sample fluid is flowed over the test surface, until the time when the last volume of buffer fluid is flowed over the test surface; zeroing the parts of the binding curve which not in said plurality predefined interval time periods; and using only said parts of the binding curve which are not zeroed to determine the kinetic parameters. Optionally, the part of the binding curve which is after the last volume of sample fluid has flowed over the test surface until the level of binding drops to a predefined threshold level may not be zeroed.

As was the case for the first and second embodiments, preferably the buffer fluid has a composition which will enable the buffer to promote the dissociation of analytes, which were bound to the ligands on the test surface, from said ligands. Most preferably the respective volumes of sample fluid preferably are each composed of a buffer fluid comprising analyte; and the respective volumes of buffer fluid preferably are each composed of the same buffer fluid as is in the volumes of sample fluid, but without the analyte.

The step of using only the parts of the binding curve which are which are in said plurality of predefined interval time periods (and optionally, also the part of the binding curve which is after the last volume of sample fluid has flowed over the test surface until the level of binding drops to a predefined threshold level), to determine the kinetic parameters, may be done in the same manner as described above in the first or second embodiments the only difference been that in the first and second embodiments only two volumes of sample fluid (first and second volumes of sample fluid (V1,V2)) and only two interval time periods (first interval time period (t1Δ), and second interval time period (t2Δ), are used, whereas in this second embodiment this concept is extended to multiple volumes of sample fluid and a plurality of predefined interval time periods.

The step of using only the parts of the binding curve which are in said plurality of predefined interval time periods (and optionally, also the part of the binding curve which is after the last volume of sample fluid has flowed over the test surface until the level of binding drops to a predefined threshold level) to determine the kinetic parameters, may be carried out using any known technique for determining kinetic parameters using a binding curve; the very same techniques can be used in the present invention to determine kinetic parameters, the difference being the techniques are applied exclusively to the parts of the binding curve which are in said plurality of predefined interval time periods (and optionally, also the part of the binding curve which is after the last volume of sample fluid has flowed over the test surface until the level of binding drops to a predefined threshold level) only.

Most preferably, in order to determine the kinetic parameters only the parts of the binding curve which are in said plurality of predefined interval time periods (and optionally, also the part of the binding curve which is after the last volume of sample fluid has flowed over the test surface until the level of binding drops to a predefined threshold level), the following steps are carried out: establishing a normalized concentration curve (c(t)) which describes the concentration of the analyte at the test surface over time (using any of the techniques described in the present application); estimating values for the kinetic parameters Rmax, kₐ, k_{d}, wherein Rmax is a predefined theoretical maximum binding curve value corresponding to a saturation of the ligands, such as for instance when all binding sites of the ligands are occupied by analyte bound to the ligands, kₐ is the association rate constant and k_{d} is the dissociation rate constant; using said normalized concentration curve (c(t)) determined in said calibration step, and said estimated values for the kinetic parameters Rmax, kₐ, k_{d}, to solve the differential reaction equation:

$$\frac{d R}{d t} = {k}_{a} c \left(t\right) \left({R}_{m a x} - R \left(t\right)\right) - {k}_{d} R \left(t\right)$$

in order to obtain a simulated binding curve.

Once the differential reaction equation has been obtained, then extracting the parts of the simulated binding curve which are in said plurality of predefined interval time periods (tjΔ) to provide respective partial simulated binding curves (sbcj).

A partial chi square (χ²) of the simulated binding curve is then established according to the following equation:

$${\text{χ}}^{2} = {\sum }_{j} {\sum }_{i} \frac{{\left({\text{sbcj}}_{i} {\text{-dmbj}}_{i}\right)}^{2}}{{N}_{d m b j}}$$

wherein dmbj is the part of the binding curve which is in the j-th interval time period (tjΔ), and wherein sbcj is the j-th partial simulated binding curve and N_{dmbj} is the number of data points in the part of the binding curve which is in the j-th predefined interval time period (tjΔ).

After the partial chi square (χ²) of the simulated binding curve has been determined then, minimizing said determined partial chi square (χ²), wherein the values of said kinetic parameters Rmax, kₐ, k_{d} which minimize said determined partial chi square (χ²) define the kinetic parameters of said reaction between the analyte and ligands which are attached to a test surface of the flow cell.

In the preferred embodiment the step of estimating values for the kinetic parameters Rmax, kₐ, k_{d}, comprises using a Levenberg-Marquard algorithm to find the kinetic parameters which minimize the partial chi square (χ²).

In a further preferred embodiment the step of estimating values for the kinetic parameters Rmax, kₐ, k_{d}, comprises using Estimators to find the kinetic parameters which minimize the partial chi square (χ²).

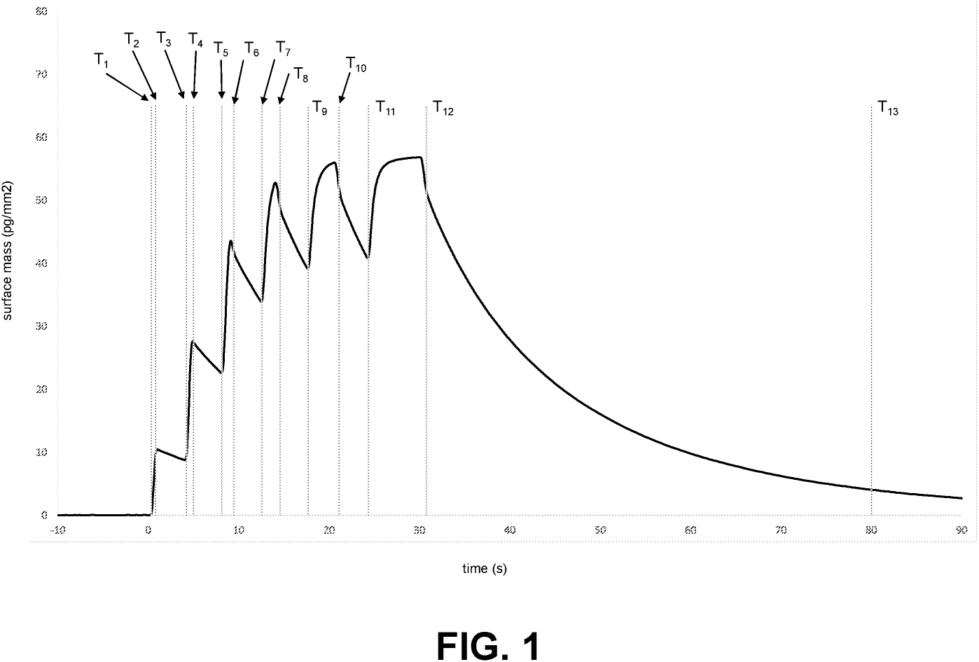

### Description of FIGS. 1-3:

FIGS. 1-3 can be used to demonstrate the principles outlined in the second embodiment above, being applied when multiple number of volumes of sample fluid are flowed over the test surface of the flow cell.

FIG. 1 shows an example of complete binding curve which is obtained when six volumes of sample fluid (V1-V6) are consecutively flowed over the test surface of the flow cell. The complete binding curve shows the amount of analyte which is bound to the ligands on the test surface as a first volume of sample fluid (V1) containing analyte is flowed over the test surface (T1-T2); after the first volume of sample fluid containing analyte is flowed over the test surface and before the a second volume of sample fluid is flowed over the test surface (T2-T3), during the time period T2-T3, a first volume of buffer fluid (V′1) (which does not contain analyte) is flowed over the test surface; as a second volume of sample fluid (V2) containing analyte is flowed over the test surface (T3-T4); after the second volume of sample fluid containing analyte is flowed over the test surface and before the a third volume of sample fluid is flowed over the test surface (T4-T5), during the time period T4-T5, a second volume of buffer fluid (V′2) (which does not contain analyte) is flowed over the test surface; as a third volume of sample fluid (V3) containing analyte is flowed over the test surface (T5-T6); after the third volume of sample fluid containing analyte is flowed over the test surface and before the a fourth volume of sample fluid is flowed over the test surface (T6-T7), during the time period T6-T7, a third volume of buffer fluid (V′3) (which does not contain analyte) is flowed over the test surface; as a fourth volume of sample fluid (V4) containing analyte is flowed over the test surface (T7-T8); after the fourth volume of sample fluid containing analyte is flowed over the test surface and before the a fifth volume of sample fluid is flowed over the test surface (T8-T9), during the time period T8-T9, a fourth volume of buffer fluid (V′4) (which does not contain analyte) is flowed over the test surface; as a fifth volume of sample fluid (V5) containing analyte is flowed over the test surface (T9-T10); after the fifth volume of sample fluid containing analyte is flowed over the test surface and before the a sixth volume of sample fluid is flowed over the test surface (T10-T11) during the time period T10-T11, a fifth volume of buffer fluid (V′5) (which does not contain analyte) is flowed over the test surface; as a sixth volume of sample fluid (V6) containing analyte is flowed over the test surface (T11-T12); after the sixth volume of sample fluid containing analyte is flowed over the test surface, until the amount of analyte which is bound to the ligands reduced to a predefined threshold level or until a predefined time period has elapsed (T12-T13), preferably during the time period T12-T13 a sixth volume of buffer fluid (V′6) (which does not contain analyte) is flowed over the test surface .

Preferably the volumes of buffer fluid (V′1-V′6) may preferably have a composition which allows the buffer fluid to promote the dissociation of analytes, which were bound to the ligands on the test surface, from said ligands. Most preferably each of the volumes of sample fluid (V1-V6) are composed of buffer fluid mixed with analyte; each of the volumes of buffer fluid (V′1-V′6) are composed of buffer fluid only (i.e. without the analyte), and most preferable the buffer fluid in each of the volumes of buffer fluid (V′1-V′6) is the same as the buffer fluid in the volumes of sample fluid (V1-V6).

Each of the six volumes of sample fluid (V1-V6) have a known concentration of analyte. The concentration of analyte in each volume can be determined using any known means in the art. Thus the concentration of analyte in each of the six volumes of sample fluid (V1-V6) is known. The volume of sample fluid (V1-V6) with the largest concentration of the analyte is the maximum concentration (cₘₐₓ). In this example each of the six volumes of sample fluid (V1-V6) happen to have the same concentration of analyte (so each volume of sample fluid has the a concentration of analyte which equal to the maximum concentration (cₘₐₓ)); however in another embodiment the six volumes of sample fluid (V1-V6) may have different concentrations of analyte in which case the maximum concentration (cₘₐₓ) is the concentration of analyte in the volume of sample fluid (V1-V6) with the largest concentration.

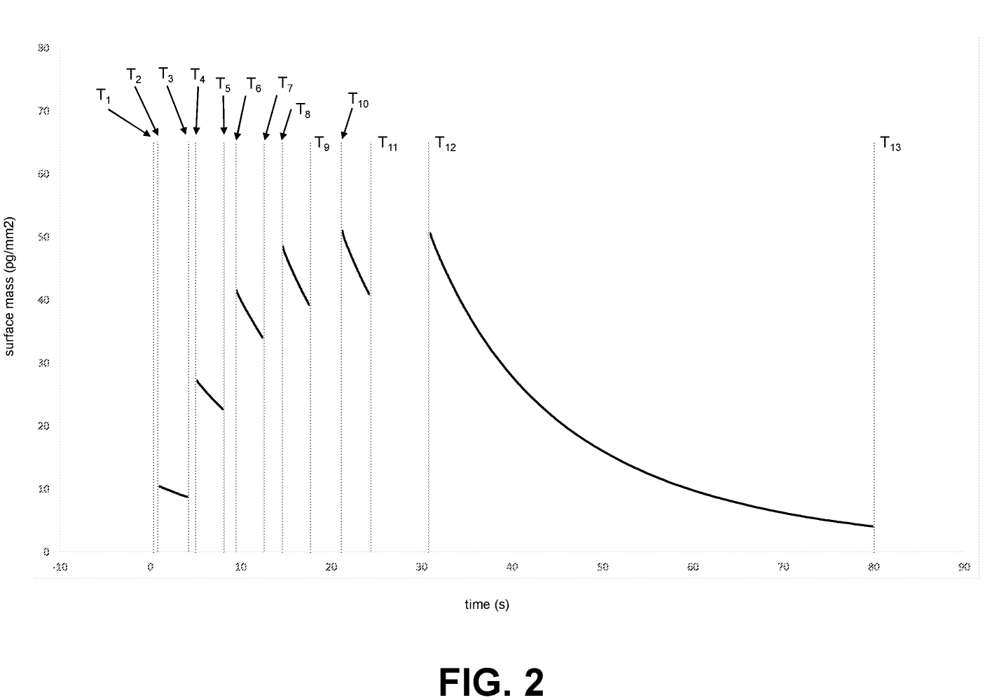

FIG. 2 shows a plurality of predefined interval time periods of the binding curve of FIG. 1 extracted. It should be understood that FIG. 2 could alternatively be considered to show a plurality of predefined interval time periods of the binding curve of FIG. 1, with the other parts of the binding curve have been ‘zeroed’. Either way, in the present invention FIG. 2 illustrates the part of the binding curve which are used, exclusively, in the present invention to determine the kinetic parameters (e.g. in step (e) of the first embodiment).

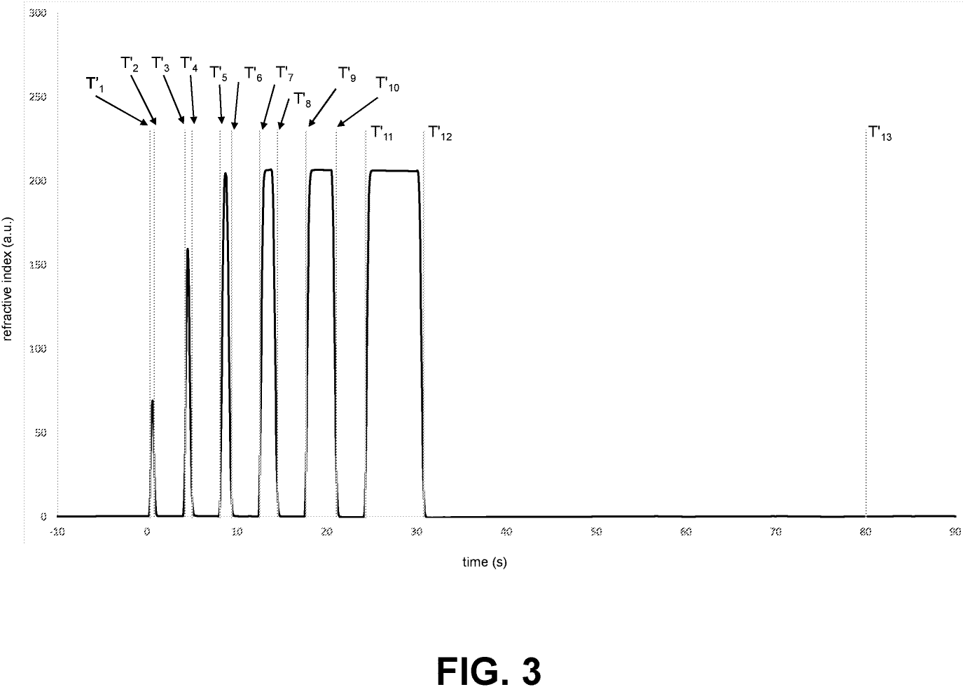

It should be understood that the predefined interval time periods are determined, preferably from a concentration curve or from a normalized concentration curve. FIG. 3 shows a normalized concentration curve which was used to determine the predefined interval time periods; these predefined interval time periods need to be determined to know which parts of the binding curve of FIG. 1 to extract in order to form the curve shown in FIG. 2.

In this case six volumes of sample fluid (V1-V6) are consecutively flowed over the test surface of the flow cell, in order to establish a binding curve (FIG. 1), so a corresponding six volumes of refractive index standard fluid, which contains a known concentration of reference molecules, are flowed over the test surface in order to obtain the normalized concentration curve in FIG. 3.

The normalized concentration curve in FIG. 3 obtained after, a first volume of refractive index standard fluid (V″1) is flowed over the test surface; after the first volume of refractive index standard fluid is flowed over the test surface and before the a second volume of refractive index standard fluid is flowed over the test surface, during this time period, a first volume of buffer fluid (V′1) (which does not contain reference molecules) is flowed over the test surface; as a second volume of refractive index standard fluid (V″2) is flowed over the test surface; after the second volume of refractive index standard fluid is flowed over the test surface and before the a third volume of refractive index standard fluid is flowed over the test surface, during this time period, a second volume of buffer fluid (V′2) (which does not contain reference molecules) is flowed over the test surface; as a third volume of refractive index standard fluid (V″3) is flowed over the test surface; after the third volume of refractive index standard fluid is flowed over the test surface and before the a fourth volume of refractive index standard fluid is flowed over the test surface, during this time period, a third volume of buffer fluid (V′3) (which does not contain reference molecules) is flowed over the test surface; as a fourth volume of refractive index standard fluid (V″4) is flowed over the test surface; after the fourth volume of refractive index standard fluid is flowed over the test surface and before the a fifth volume of refractive index standard fluid is flowed over the test surface, during this time period a fourth volume of buffer fluid (V′4) (which does not contain reference molecules) is flowed over the test surface; as a fifth volume of refractive index standard fluid (V″5) is flowed over the test surface; after the fifth volume of refractive index standard fluid is flowed over the test surface and before the a sixth volume of refractive index standard fluid is flowed over the test surface, during this time period, a fifth volume of buffer fluid (V′5) (which does not contain reference molecules) is flowed over the test surface; as a sixth volume of refractive index standard fluid (V″6) is flowed over the test surface; after the sixth volume of refractive index standard fluid is flowed over the test surface, until the concentration of the reference molecule at the test surface reduces to a predefined threshold level or until a predefined time period has elapsed, preferably during this time period a sixth volume of buffer fluid (V′6) (which does not contain reference molecules) is flowed over the test surface. Measuring the concentration of reference molecules at the test surface while each of the afore-mentioned steps are carried out (preferably from before the first volume of sample fluid (V1) is flowed over the test surface, until the end of last step when the sixth volume of buffer fluid (V′6) is flowed over the test surface until the concentration of the reference molecule at the test surface reduces to a predefined threshold level or until a predefined time period has elapsed, results in a concentration curve (cmc).

Importantly the rate at which the respective volumes of refractive index standard fluid are flowed over the test surface (i.e. the flow rate) is equal to the rate at which the respective six volumes of sample fluid (V1-V6) are flowed over the test surface. Also the ratio of the concentrations of the volumes of refractive index standard fluid (V1″-V″6) is equal to the ration of the concentrations of the volumes of sample fluids (V1-V6).

The concentration curve (cmc) is then normalized by dividing the concentration curve (cmc)) by the maximum value of the concentration curve (max(cmc)) and multiplying the result by the known maximum concentration (cₘₐₓ) of analyte in the respective six volumes of sample fluid (V1-V6) as illustrated in the following equation:

$$\text{c} \left(\text{t}\right) = \left(\left(\text{cmc}\right) / \text{max} \left(\text{cmc}\right)\right) * {\text{c}}_{max}$$

to provide a normalized concentration curve (c(t)) shown in FIG. 3.

In order to determine each of the plurality of predefined interval time periods from the normalized concentration curve (c(t)) shown in FIG. 3. A threshold concentration is selected; in this example the threshold concentration is 5% of the maximum value of the normalized concentration curve (c(t)); since the maximum value of the normalized concentration curve (c(t)) is ‘200’, in this example the threshold concentration is ‘10’ (i.e. 5%*200 = 10). The time intervals where the normalized concentration curve (c(t)), drops to below the level of ‘10’, until is raises again to be equal to, or above, ‘10’ defines the plurality of predefined interval time periods. Referring to the normalized concentration curve (c(t)) shown in FIG. 3: at time T′₂ the normalized concentration curve (c(t)), drops to below the level of ‘10’, at time T′₃ the normalized concentration curve (c(t)), raises to ‘10, thus T′₂-T′₃ defines one of the predefined interval time periods; at time T′₄ the normalized concentration curve (c(t)), drops to below the level of ‘10’, at time T′₅ the normalized concentration curve (c(t)), raises to ‘10, thus T′₄-T′₅ defines another one of the predefined interval time periods; at time T′₆ the normalized concentration curve (c(t)), drops to below the level of ‘10’, at time T′₇ the normalized concentration curve (c(t)), raises to ‘10, thus T′₆-T′₇, defines another one of the predefined interval time periods; at time T′₈ the normalized concentration curve (c(t)), drops to below the level of ‘10’, at time T′₉ the normalized concentration curve (c(t)), raises to ‘10, thus T′₈-T′₉ defines another one of the predefined interval time periods; at time T′₁₀ the normalized concentration curve (c(t)), drops to below the level of ‘10’, at time T′₁₁ the normalized concentration curve (c(t)), raises to ‘10, thus T′₁₀-T′₁₁ defines another one of the predefined interval time periods; at time T′₁₂ the normalized concentration curve (c(t)), drops to below the level of ‘10’, at time T′₁₃ a predefined time period has elapsed, thus T′₁₂-T′₁₃ defines the last of the predefined interval time periods.

The parts of the binding curve shown in FIG. 1, which occur at times corresponding to the predefined interval time periods T′₂-T′₃, T′₄-T′₅, T′₆-T′₇, T′₈-T′₉,T′₁₀-T′₁₁, T′₁₂-T′₁₃ are then extracted to provide the binding curve shown in FIG. 2, which is used to determine the kinetic parameters Rmax, kₐ, k_{d} in the manner describe above in each of the embodiments of the present invention.

Various modifications and variations to the described embodiments of the invention will be apparent to those skilled in the art without departing from the scope of the invention as defined in the appended claims. Although the invention has been described in connection with specific preferred embodiments, it should be understood that the invention as claimed should not be unduly limited to such specific embodiment.

1. A method for determining the kinetic parameters of a reaction between an analyte and ligands which are attached to a test surface of a flow cell, the method comprising the steps of,
(a) flowing a first volume of sample fluid (V1), which contains said analyte, over the test surface, between a first point in time (t₁) to a second point in time (t₂);
(b) flowing a first volume of buffer fluid (Vb1), which is without analyte, over the test surface, between a third point in time (t₃) to a fourth point in time (t₄);
(c) flowing at least a second volume of sample fluid (V2), which contains said analyte, over the test surface, between a fifth point in time (t₅) to a sixth point in time (t₆);
(d) flowing at least a second volume of buffer fluid (Vb2), which is without analyte, over the test surface, between a seventh point in time (t₇) to a eight point in time (t₈);
(e) using a sensor to measure the binding of analyte to ligands on the test surface to obtain a binding curve;
(f) using the only the parts of the binding curve which are in predefined interval time periods, without using the other parts of the binding curve, to determine the kinetic parameters.

2. A method according to claim 1 wherein the predefined interval time periods comprise a first and second interval time periods (t1Δ, t2Δ);
wherein the first interval time period (t1Δ) occurs between the second point in time (t₂) and fifth point in time (t₅); and wherein the second interval time periods (t2Δ) occurs between the sixth point in time (t₆) and a ninth point in time (t₉) wherein the ninth point in time (t₉) occurs sometime after the eight point in time (t₈), or, the ninth point in time (t₉) is equal to the eight point in time (t₈)).

3. A method according to claim 2, wherein the first interval time period (t1Δ) is a portion of the duration of time between the second point in time (t₂) and the fifth point in time (t₅); and the second interval time period (t2Δ) is a portion of the duration of time between the sixth point in time (t₆) and a ninth point in time (t₉); or
wherein the first interval time period (t1Δ) is defined by the whole time interval between the second point in time (t₂) and the fifth point in time (t₅); and the second interval time period (t2Δ) is defined by the whole time interval between the sixth point in time (t₆) and a ninth point in time (t₉).

4. A method according to claim 1 further comprising a calibration step to determine the predefined interval time periods;
wherein the calibration step comprises, establishing a concentration curve; and using said concentration curve to determine the predefined interval time periods.

5. A method according to claim 4 wherein the step of using said concentration curve to determine the predefined interval time periods, comprises,
selecting a threshold concentration;
identifying the time instants when the concentration curve is equal to the to the threshold concentration;
wherein each respective predefined interval time period is defined by a period between a time instant when the concentration curve is at the threshold concentration, said concentration curve showing a decrease in concentration before said time instant, and, a next time instant when the concentration curve is at the threshold concentration, said concentration curve showing an increase in concentration before said next time instant.

6. A method according to claim 5 wherein the step of selecting a threshold concentration comprises,
identifying the maximum value of the concentration curve;
selecting a percentage value, wherein the threshold concentration is defined by the maximum value of the concentration curve multiplied by said percentage value.

7. A method according to claim 1 further comprising a calibration step to determine the predefined interval time periods;
wherein the calibration step comprises, establishing a concentration curve; normalizing said concentration curve to provide a normalized concentration curve (c(t)); and then using that normalized concentration curve (c(t)) to determine the predefined interval time periods.

8. A method according to claim 7 wherein the step of using said normalized concentration curve to determine the predefined interval time periods, comprises,
selecting a threshold concentration;
identifying the time instants when the normalized concentration curve is equal to the to the threshold concentration;
wherein each respective predefined interval time period is defined by a period between a time instant when the normalized concentration curve is at the threshold concentration, said normalized concentration curve showing a decrease in concentration before said time instant, and, a next time instant when the normalized concentration curve is at the threshold concentration, said normalized concentration curve showing an increase in concentration before said next time instant.

9. A method according to claim 8 wherein the step of selecting a threshold concentration comprises,
identifying the maximum value of the normalized concentration curve; selecting a percentage value, wherein the threshold concentration is defined by the maximum value of the normalized concentration curve multiplied by said percentage value.

10. A method according to claim 1 comprising,
using a sensor to measure the binding of analyte to ligands on the test surface, continuously from the first point in time (t₁), or before the first point in time (t₁), until the eight point in time (t₈) or after the eight point in time (t₈); extracting the parts of the binding curve which are in said predefined interval time periods;
using only said extracted parts of the binding curve to determine the kinetic parameters.

11. A method according to claim 1 comprising,
using a sensor to measure the binding of analyte to ligands on the test surface, continuously from the first point in time (t₁), or before the first point in time (t₁), until the eight point in time (t₈) or after the eight point in time (t₈); zeroing the parts of the binding curve which are outside of said predefined interval time periods;
using only said parts of the binding curve which are not zeroed to determine the kinetic parameters.

12. A method according to claim 1 comprising the steps of,
consecutively flowing a plurality of flowing a plurality of volumes of sample fluid, each of which contains said analyte, over the test surface, wherein there is an interval time period between each respective volume of sample fluid is flowed over the test surface;
flowing a respective volume of buffer fluid, which is without analyte, over the test surface during each respective interval time period;
using a sensor to measure the binding of analyte to ligands on the test surface, to obtain a binding curve;
using only parts of the binding curve which are in a plurality of predefined interval time periods, to determine the kinetic parameters.

13. A method according to claim 1, wherein the step of using the only the parts of the binding curve which are in predefined interval time periods, without using the other parts of the binding curve, to determine the kinetic parameters, comprises the steps of,
establishing a normalized concentration function c(t), which describes the concentration of the analyte at the test surface over time;
estimating values for kinetic parameters Rmax, kₐ, k_{d}, wherein Rmax is a predefined maximum response value corresponding to a saturation of the ligands on the test surface, kₐ is the association rate constant and k_{d} is the dissociation rate constant;
using said normalized concentration curve (c(t)) and the estimated values for the kinetic parameters Rmax, kₐ, k_{d}, solve the differential reaction equation:
$$\frac{d R}{d t} = {k}_{a} c \left(t\right) \left({R}_{m a x} - R \left(t\right)\right) - {k}_{d} R \left(t\right)$$
in order to obtain a simulated binding curve;
extracting parts of the simulated binding curve which are in said plurality of predefined interval time periods (tjΔ) to provide respective partial simulated binding curves (sbcj);
determining a partial chi square (_{X}²) of the simulated binding curve according to the following equation:
$${χ}^{2} = {\sum }_{j} {\sum }_{i} \frac{{\left({\text{sbcj}}_{i} - {\text{dmbj}}_{i}\right)}^{2}}{{N}_{d m b j}}$$
wherein dmbj is the part of the binding curve which is in the j-th predefined interval time period (tjΔ), and wherein sbcj is the j-th partial simulated binding curve and N_{dmbj} is the number of data points in the part of the binding curve which is in the j-th predefined interval time period (tjΔ);
minimizing said determined partial chi square (χ²), wherein the values of said kinetic parameters Rmax, kₐ, k_{d} which minimize said determined partial chi square (_{X}²) define the kinetic parameters of said reaction between the analyte and ligands which are attached to a test surface of the flow cell.

14. A method according to claim 13 wherein the step of establishing a concentration function c(t), which describes the concentration of the analyte at the test surface over time, comprises using a Levenberg-Marquard algorithm to find the kinetic parameters which minimize the partial chi square (χ²).

15. A method according to claim 13 wherein the step of estimating values for the kinetic parameters Rmax, kₐ, k_{d}, comprises using Estimators to find the kinetic parameters which minimize the partial chi square (χ²).
